(19) United States
(12) Patent Application Publication
(10) Pub. No.: US 2014/0276025 A1
(43) Pub. Date: Sep. 18, 2014

(54) MULTIMODAL INTEGRATION OF OCULAR DATA ACQUISITION AND ANALYSIS

(71) Applicant: CARL ZEISS MEDITEC, INC., DUBLIN, CA (US)

(72) Inventors: Mary K. DURBIN, San Francisco, CA (US); Utkarsh SHARMA, San Leandro, CA (US); Harihar NARASIMHA-IYER, Livermore, CA (US); Martin HACKER, Jena (DE); Allen JONES, Dublin, CA (US); Christine N. RITTER, Alamo, CA (US)

(73) Assignee: CARL ZEISS MEDITEC, INC., Dublin, CA (US)

(21) Appl. No.: 14/207,060

(22) Filed: Mar. 12, 2014

Related U.S. Application Data
(60) Provisional application No. 61/785,420, filed on Mar. 14, 2013.
(60) Provisional application No. 61/934,114, filed on Jan. 31, 2014.

(51) Int. Cl. A61B 5/00; A61B 3/10

(52) U.S. Cl. CPC A61B 5/4842; A61B 3/102
USPC 600/427; 600/407

(57) ABSTRACT

Regions-of-interest discovered from analyses of images obtained from one imaging modality can be further observed, analyzed, supplemented, and further analyzed by one or more additional imaging modalities and in an automated way. In addition, one or more pathologies identified from analyses of these regions-of-interest, and a metric of the likelihood of the presence of disease, and/or a metric of risk of disease progression can be derived therefrom.

### CROSS-REFERENCE TO RELATED APPLICATIONS

The present application claims benefit of U.S. provisional applications with Ser. Nos. 61/785,420, filed Mar. 14, 2013, and 61/934,114, filed Jan. 31, 2014, and are incorporated herein by reference in their entirety.

### FIELD OF THE INVENTION

This application presents methods of using information derived from one ophthalmic imaging modality to guide acquisition and analysis using a second imaging modality. The information content of the various modalities can yield estimates on the degree of disease progression.

### BACKGROUND

### Introduction

There are various imaging modalities of the interior of an eye from which important diagnostic information regarding the state of health of the eye can be derived. These modalities include, but are not limited to, optical coherence tomography (OCT) which includes the various imaging modalities of OCT including its functional extensions (Doppler, fluorescein angiography, contrast agents, oximetry, fluorophores, phase-sensitive, polarization sensitive, spectroscopic); fundus imaging which includes fundus cameras, stereo imaging devices, confocal scanning laser ophthalmoscopes (cSLO), line scanning ophthalmoscopes (LSO), fluorescein angiography (FA), fundus biomicroscopy, fundus autofluorescence (FAF), and broad-line fundus imagers (BLFI). The information content of data obtained from each of these modalities is not necessarily duplicated by another of these modalities. Thus combining the information derived from various modalities can yield important clues as to the diagnosis and prognosis of disease and its implications.

### Structural OCT

Optical Coherence Tomography (OCT) is a technology for performing high-resolution cross sectional imaging that can provide three dimensional images of tissue structure on the micron scale in situ and in real-time. OCT is a method of interferometry that uses light containing a range of optical frequencies to determine the scattering profile of a sample. The axial resolution of OCT is inversely proportional to the span of optical frequencies used.

OCT technology has found widespread use in ophthalmology for imaging different areas of the eye and providing information on various disease states and conditions. In addition to collecting data at different depths or locations, different scan patterns covering different transverse extents can be desirable depending on the particular application.

OCT has the ability to image the different retinal tissues such as the internal limiting membrane (ILM), nerve fiber layer (NFL or RNFL), retinal pigment epithelium (RPE), ganglion cell complex or layer (GCC or GCL), Bruch's membrane, inner segments (IS), outer segments (OS), and the choroid. Moreover, with OCT data, segmentation of not only the aforementioned retinal layers and others as well, but the segmentation and further analyses of morphological pathologies such as, e.g., drusen and geographic atrophy also augment the usefulness of this modality. (See, e.g., Gregori et al. 2011; Yehoshua et al. 2013.)

Moreover, it permits the ability to identify many retinal pathological areas such as macular edema, macular detachment, macular hole, central serous retinopathy, and elevated RPE. In the last case, often referred to as pigment epithelial detachment (or PED), the cause may be serous fluid, fibrovascular tissue, hemorrhage, or the coalescence of drusen beneath the RPE. Although PEDs can occur in the context of non-neovascular age-related macular degeneration, most, however, are related to choroidal neovascularization (CNV). This neovascularization can spread and cause fluid accumulation away from the CNV to create a serous PED. (Thus it is considered that PED's are at least a subset of problems associated with RPE elevation.)

### Functional OCT

Functional OCT can provide important clinical information that is not available in the typical intensity based structural OCT images. There have been several functional contrast enhancement methods including Doppler OCT, Phase-sensitive OCT measurements, Polarization Sensitive OCT, Spectroscopic OCT, nanoparticle contrast-enhanced OCT, second harmonic generation OCT, etc. Integration of functional extensions can greatly enhance the capabilities of OCT for a range of applications in medicine. One of the most promising functional extensions of OCT has been the field of OCT angiography which is based on flow or motion contrast. The field of OCT angiography has generated a lot of interest in the OCT research community during the last few years. There are several flow contrast techniques in OCT imaging that utilize inter-frame change analysis of the OCT intensity or phase-resolved OCT data (see, e.g. Wang et al. 2007; An & Wang 2008; Fingler et al. 2007; Fingler et al. 2009; Mariampillai et al. 2010; Fingler et al. 2008 in US20080025570; and U.S. Pat. No. 8,433,393).

One of the major applications of such techniques has been to generate en face vasculature images of the retina. En face images are typically generated from three dimensional data cubes by summing pixels along a given direction in the cube, either in their entirety or from sub-portions of the data volume (see for example U.S. Pat. No. 7,301,644). Visualization of the detailed vasculature using functional OCT enables doctors to obtain new and useful clinical information for diagnosis and management of eye diseases in a non-invasive manner.

The family of optical coherence tomographic systems comprising both structural and functional aims is, within the present application referred to, as optical coherence imaging modalities, optical coherence tomographic modalities, OCT imaging modalities, or optical coherence tomographic imaging modalities. The specific class of functional OCT shall be also identified as functional optical coherence tomographic systems or functional OCT. This class involves the ability to study motion and flow including but not limited to blood flow and perfusion, oxygen perfusion, metabolic processes such as consumption of energy, conversion of glucose into ATP, utilization of ATP especially by the mitochondria, and the like.

Diagnostic Information from OCT

OCT characteristic information derivable from the aforementioned OCT imaging modalities (or optical coherence imaging modalities) include, but are not limited to: thicknesses of the various retinal layers; volumetric information regarding drusen (3D size)—an early indicator of age-related macular degeneration; extent of retinal thickening or the hard exudates associated therewith; the extent of diabetic macular edema; extent of macular edema due to retinal vein occlusion; extent of diseases of the vitreomacular interface such as epiretinal membranes; the extent of macular holes, pseudoholes, schisis from myopia or optic pits; the extent of serous chorioretinopathy; the extent of retinal detachment; extent of blood flow in the retina; the extent of vascular perfusion or lack thereof; and with repeated measurements of a similar kind, chronological changes that can help suggest prognosis or progression.

### Variations on a Theme of Fundus Imaging

Fundus imaging of the eye is basically a 2D projection of the 3D retina using light reflected off the retina. The light can be monochromatic or polychromatic, depending upon the desire to enhance certain features or depths. There are various instrumental approaches to what amounts to fundus. These include, but are not limited to, fundus cameras, scanning laser ophthalmoscopes (SLO), line scanning ophthalmoscopes (LSO), biomicroscopy, fluorescein (FA) or indocyanian green (ICG) angiography, scanning laser polarimetry (SLP), fundus autofluorescence (FAF), confocal scanning laser ophthalmoscopes (cSLO), and broad line fundus imaging (BLFI). Variety of wavelengths can be used in the scanning beam (NIR, color, RGB, RGB-splits). Stereo fundus imaging is obtainable via combining separate images taken at different angles. FA could also be achieved by taking sequential images (i.e., FA movie or movies). A live FA image is also possible (OPMI-display).

The highest contrast modality of fundus imaging is that obtained using a confocal scanning laser ophthalmoscope, in which every point is illuminated by a single laser and the reflected light at a certain selected depth is allowed to pass through a small aperture which blocks light from other depths. The images have excellent lateral and axial resolutions as well as good contrast between structures being imaged.

Several of the aforementioned fundus imaging modalities are of a functional nature, which permit understandings or insight into neuroanatomical basis of psychophysical and pathophysiological phenomena.

Use of reflectance based fundus imaging such as fundus camera, confocal scanning laser ophthalmoscopes (cSLO), line scanning ophthalmoscopes (LSO), and broad-line fundus imagers (BLFI) can also generate some functional information such as blood flow (see for example, Ferguson et al. 2004).

Functional observations can include detection of ischemic regions, evaluation of biochemical changes associated with various pathological conditions, localization of drugs and efficacy thereof, blood flow, glucose utilization, oxygen utilization, and other metabolic processes and molecules are to name just a few.

Fluorescein or indocyanine green angiography are modes of functional fundus imaging, which use fluorophores that are injected into the blood stream of a patient. As time progresses, these fluorophores reach the blood vessels of the eye. Subsequently, upon examination of the retina of an eye within a certain wavelength band, the circulation pattern can be observed due to the emission from the photon-stimulated fluorophores.

Another functional mode is that of fundus autofluorescence and is based on the fluorescence of lipofuscin in the retinal pigment epithelium (hereinafter, RPE). Lipofuscin is a residue of phagocytosed photoreceptor outer segments. FAF's principal use is in detecting pathological changes in the RPE, which include, but are not limited to, macular pigments, photopigments, and macrophages in the subretinal space.

FAF is also a popular method for imaging of geographic atrophy (GA), which is characterized by the loss of various retinal layers, including outer nuclear layer, external limiting membrane, inner and even outer segments of photoreceptors, down to the RPE. This pathological disturbance is a morphological appearance identified via hypopigmentation/-depigmentation due to the absence of the retinal pigment epithelium. Depending on the wavelength of light used for stimulation, autofluorescence images may suffer from loss of signal near the fovea, a problem that does not occur in OCT visualization of GA. Certain patterns of autofluorescence at the margin of GA have been shown to correlate with faster progression of the pathologies associated with GA. OCT also shows different patterns of retinal layer disruption at the borders of geographic atrophy (Brar et al. 2009), and those patterns of disruption have been shown to be related to patterns of hyperautofluorescence (Sayegh et al. 2011).

The term ‘fundus imaging’ will be referred to hereinafter as any aforementioned system to image the fundus of an eye (see, e.g., Abramoff et al. 2010). The class of functional fundus imaging modalities refers to FA, ICG, Doppler, oximetry, FAF, and any other mode which measures blood flow or perfusion, oxygen flow or perfusion, metabolic processes, consumption of energy, conversion of glucose into ATP, utilization of ATP especially by mitochondria, activity of lysosomes, oxidation of fatty acids, and the like.

### Fundus-Guided OCT Imaging

Ophthalmologists often recognize suspect retinal features by reviewing and analysing fundus imagery (color, FAF, FA, ICG, RGB-splits, stereo), for example pigmentation changes or abnormalities (color images, RGB), functional distortions in the vessel system such as in diabetic retinopathy, retinal ischemia, neovascularization (FA, ICG) or other metabolic abnormalities or atrophies (FAF). For more specific diagnosis and treatment guidance, additional structural information from the exact location of the features is desired, for example, high-resolution OCT B-scans (see, e.g., US2007029177) to show internal structural details in the area of the abnormality. In addition, OCT may be used to extract functional information such as blood flow that may provide additional information.

Pemp et al. (2013) recently concluded in a study that image quality and reproducibility of mean peripapillary RNFLT measurements using SD-OCT is improved by averaging OCT images with eye-tracking compared to un-averaged single frame images. While they used tracking to compare the repeatability and changes, the baseline circle scan was placed manually. The biggest drawback of this method is that manual placement of a circle is susceptible to operator error and wrong placement of the circles creates the difficulty in comparing the TSNIT (temporal-superior-nasal-inferior-temporal) thickness with normative databases.

Diagnostic Information from Fundus Imaging

Fundus characteristic information derivable from fundus imaging modalities include, but are not limited to: extent of drusen, geographic atrophy, hard and soft exudates, cotton-wool spots, blood flow, ischemia, vascular leakage, reflectivities as a function of depth and wavelength; hyper- or hypo-pigmentation abnormalities (often due to the absence of melanin or the presence of lipofuscin); colors based on relative intensities at different wavelengths; and chronological changes in any of these. The extent of many of these observables is directly correlated with the likelihood of the presence of disease, as is well known in the art.

For the purposes of the present application, the term functional imaging or functional imaging modality shall refer to any of the aforementioned functional imaging modalities, whether it be under the rubric of optical coherence tomography imaging or within the rubric of fundus imaging.

### SUMMARY

It is the purpose of this application, to present methodologies to optimize the selection of information content from a subsequent imaging modality based upon that information derived from a first imaging modality, and, moreover, to do so in an automatic approach. In addition, the collective body of information derived from the various modalities or a subset thereof can be used to estimate the likelihood of the risk of disease progression. Thus, the information derived from one imaging modality can then be used to guide the acquisition or analysis of a subsequent imaging modality or both modalities can be analyzed together. This could be accomplished on a single multimodality imaging system, or preferably via a network of imaging systems and review stations. The approach can include change analysis by imaging the same areas with the same instrument type and using the change to derive the data collection or analysis of the other modality.

Fundus imaging is the primary method for identifying intra-retinal micro-aneurysms. The accuracy of diagnosis can be enhanced by using supplementary OCT information. Functional OCT techniques such as OCT angiography can be used to detect micro-aneurysms and other vasculature abnormalities in the retina and choroids. For example, in one of the embodiments, after identifying suspected micro-aneurysms in fundus images (color, FA), OCT may be used to identify the layers where they are located. In addition, the 3D OCT structural information as well as functional OCT information can further assist in detecting different forms of microaneurysms.

Identification of micro-aneurysms and other abnormalities normally seen in fundus imaging has been used to develop automated analysis (e.g., Philip et al. 2007) for screening for diabetic retinopathy, although with suboptimal sensitivity and specificity. With the use of information derived from multiple imaging modalities, and as well repeat measurements, the accuracy of diagnostic screening techniques becomes enhanced.

OCT imaging is applicable to a variety of retinal disorders. These include the choroidal neovascularization membranes, detection of detachments, including both pigment-epithelium and neurosensory, and subretinal fluids. Moreover, with the addition of the third spatial component (depth), volumetric information, unlike that derivable from 2D fundus imaging, allows thicknesses of the various retinal layers to be obtained via segmentation, and these thicknesses can be correlated with known areas of pathology. (See, e.g., US20070216909 and US20070103693.)

Analyses which could provide valuable information regarding prognosis or even likelihood of progression of disease include the segmentation of the ILM to RPE layers, the segmentation of the NFL or the ganglion cell complex (GCL or GCC), segmentation of the optical nerve head, detection of the fovea or macula, extraction of the NFL about the optic nerve head, and following automatically of the protocol of the Early Treatment of Diabetic Retinopathy Study (ETDRS). (See, e.g., Salam et al. 2013 for an explanation of the ETDRS.)

Functional OCT could further expand the capabilities of OCT to look into pathologies including wet AMD, dry AMD, diabetic retinopathy (DR), vein artery occlusions (BRVO, CRVO), ischemia, polypoidal choroidal vasculopathy (PCV), choroidal neovascularization (CNV), intraretinal microvascular abnormality (IRMA), and macular telangiectasia, just to name a few.

The information content derivable from any one of these modalities may not necessarily be duplicated by any other of the modalities. This is primarily due to the various reflective and translucent layers that make up the retina. Different imaging modalities may uses different wavelengths, lateral resolution, and depth sectioning capabilities as well as post-processing methods. The reflectance, absorption, and scattering properties of different tissues may have strong dependence on wavelength used. This means that the reflected light is not uniquely correlated with its depth within the retina. Moreover, pathological disturbances within the eye may each have a nearly unique or unique signature dependent upon the imaging modality used. Combining the information content derived from various modalities thus can provide more valuable information about the state, size or extent, origin, and likely progression of the pathology than that provided by any one modality alone.

### BRIEF DESCRIPTION OF THE DRAWINGS

FIG. 1 is a schematic of a basic Fourier-domain OCT instrument.

FIG. 2 shows a multimodal ophthalmic imaging system combining and OCT imaging modality with a line scanning ophthalmoscope, a fundus imaging modality.

FIG. 3 shows a fundus image that could be used for an embodiment of the present invention directed towards automating collection of OCT image data based on landmarks or abnormalities identified within the fundus image.

FIG. 4a shows an FA image of a subject with diabetic retinopathy. FIG. 4b is an OCT functional image showing only the fovea. The detailed visualization of the foveal avascular zone can be followed over time without contrast agent or injection.

FIG. 5 s a schematic of the various interactions between various components of an embodiment.

### DETAILED DESCRIPTION

A generalized Fourier or Frequency Domain optical coherence tomography (FD-OCT) system used to collect an OCT dataset suitable for use with the present set of embodiments, disclosed herein, is illustrated in FIG. 1. A FD-OCT system includes a light source, 101, typical sources including but not limited to broadband light sources with short temporal coherence lengths or swept laser sources.

Light from source 101 is routed, typically by optical fiber 105, to illuminate the sample 110, a typical sample being tissues at the back of the human eye. The light is scanned, typically with a scanner 107 between the output of the fiber and the sample, so that the beam of light (dashed line 108) is scanned over the area or volume to be imaged. Light scattered from the sample is collected, typically into the same fiber 105 used to route the light for illumination. Reference light derived from the same source 101 travels a separate path, in this case involving fiber 103 and retro-reflector 104. Those skilled in the art recognize that a transmissive reference path can also be used. Collected sample light is combined with reference light, typically in a fiber coupler 102, to form light interference in a detector 120. The output from the detector is supplied to a processor 121. The results can be stored in the processor or displayed on display 122. The processing and storing functions may be localized within the OCT instrument or functions may be performed on an external processing unit to which the collected data is transferred. This unit could be dedicated to data processing or perform other tasks which are quite general and not dedicated to the OCT device. The display 122 can also provide a user interface for the instrument operator to control the collection and analysis of the data.

The interference causes the intensity of the interfered light to vary across the spectrum. The Fourier transform of the interference light reveals the profile of scattering intensities at different path lengths, and therefore scattering as a function of depth (z-direction) in the sample (see, e.g., Leitgeb et al. 2004). The particular depth location being sampled at any one time is selected by setting the path length difference between the reference and sample arms to a particular value. This can be accomplished by adjusting a delay line in the reference arm, the sample arm, or both arms. Typical FD-OCT instruments can image a depth of three to four millimeters at a time.

The profile of scattering as a function of depth is called an axial scan (A-scan). A dataset of A-scans measured at neighboring locations in the sample produces a cross-sectional image (slice, tomogram, or B-scan) of the sample. A collection of B-scans collected at different transverse locations on the sample comprises a 3D volumetric dataset. Typically a B-scan is collected along a straight line but B-scans generated from scans of other geometries including circular and spiral patterns are also possible.

The sample and reference arms in the interferometer could consist of bulk-optics, fiber-optics or hybrid bulk-optic systems and could have different architectures such as Michelson, Mach-Zehnder, or common-path based designs as would be known by those skilled in the art. Light beam as used herein should be interpreted as any carefully directed light path. While an FD-OCT system has been described, aspects of the present application could be applied to any type of OCT system, including, but not limited to time-domain, spectral-domain, and swept-source. The present application also applies to systems having parallel illumination schemes, e.g., line-field and full-field.

A multimodality system that could be used with some embodiments of the present application combining an OCT scanner and a line-scan ophthalmoscope (LSO) as described in U.S. Pat. No. 7,805,009 hereby incorporated by reference is illustrated in FIG. 2. While the system illustrates an LSO, any variant of fundus imaging could be substituted.

Light from the LSO light source 201 is routed by cylindrical lens 202 and beamsplitter 203 to scanning mirror 204. The cylindrical lens 202 and the scan lens 205 produce a line of illumination at the retinal image plane 206, and the ocular lens 207 and optics of the human eye 200 re-image this line of illumination onto the retina 210. The line of illumination is swept across the retina as the scanning mirror 204 rotates. Reflected light from the retina approximately reverses the path of the LSO illumination light; the reflected light is scanned by the LSO scan mirror 204 so that the illuminated portion of the retina is continuously imaged by imaging lens 208 onto the LSO line camera 209. The LSO line camera converts the reflected LSO light into a data stream representing single-line partial images, which can be processed to form both eye tracking information and a real-time images of the retina.

The OCT system 220 incorporates the light source, light detector or detectors, and processor required to determine the depth profile of backscattered light from the OCT beam 221. The OCT system can use time or frequency domain methods. OCT scanner 222 sweeps the angle of the OCT beam laterally across the surface in two dimensions (x and y), under the control of scan controller 254. Scan lens 223 brings the OCT beam into focus on the retinal image plane 206. Beamsplitter 224 combines the OCT and LSO beam paths so that both paths can more easily be directed through the pupil of the human eye 200. (Combining the beam paths is not required in direct imaging applications, where the object itself lies in the location of the retinal image plane 206.) If the OCT and LSO use different wavelengths of light, beamsplitter 224 can be implemented as a dichroic mirror. The OCT beam is re-focused onto the retina through ocular lens 207 and the optics of the human eye 200. Some light scattered from the retina follows the reverse path of the OCT beam and returns to the OCT system 220, which determines the amount of scattered light as a function of depth along the OCT beam.

Current OCT systems typically rely on the operator to place manually the scan at the region or regions-of-interest (ROIs). This procedure is not very accurate and it is possible that the operator might miss some region of the tissue that is of interest. In addition, the fixed field-of-view of the OCT scans might miss parts of larger regions-of-interest. Unfortunately, there is no way to select a field-of-view that will work in all cases.

One of the embodiments of the present invention describes methods for automatically finding regions-of-interest based on analysis of one or more images collected from an imaging modality that is capable of generating an image of the fundus of the eye (i.e., fundus imaging modality, or en face OCT) and to adaptively change the characteristics of subsequent scans based on the information derived from the first imaging modality.

In one embodiment, OCT data are analyzed to complement/supplement the data obtainable from fundus imaging modalities. Moreover, with an ensemble of complimentary information derived from different modalities, such combined analyses could reveal extent of disease, risk of disease, or the risk or estimation of the likelihood of the progression of disease. The combined information can be then distilled into a metric of the risk of disease progression (see, e.g., Zhou et al. 2011, as has been done for glaucoma and visual field testing). An application could be for the early detection of glaucoma in which one could combine cup and disk segmentation from stereo fundus images, RNFL layer segmentation, GCC segmentation, and 3D optic disc (optic nerve head) parameters from OCT, such as the cup-to-disc ratio.

The basic embodiment described herein, is to automatically process and obtain pertinent information such as regions-of-interest upon information derived from a first imaging modality, then engaging additional imaging modalities to provide complimentary information regarding any potential pathologies located in or near the regions-of-interest. Thus providing information that can aid in elucidating the nature, extent, and progression of disease.

Such regions-of-interest might be any geometric landmark such as the fovea or the optic nerve head. They could also be areas of pathological or morphological disturbances such as subretinal fluid, macular edema, RPE elevation (which includes PED=pigment epithelial detachment), RPE tear, subretinal fibrosis, disciform scar, drusen, geographic atrophy, variations in pallor, cotton-wool spots, central serous retinopathy, wet AMD, diabetic retinopathy (DR), vein artery occlusions (BRVO, CRVO), ischemia, vascular leakage, polypoidal choroidal vasculopathy (PCV), choroidal neovascularization (CNV), intraretinal microvascular abnormality (IRMA), macular telangiectasia, retinal exudates, disc hemorrhage, and subretinal exudates.

A variety of adjunct imaging modalities such as the aforementioned varieties of fundus imaging modalities with limited imaging capabilities are known to be combined with OCT systems in order to provide a view of the fundus for use in alignment of the OCT device or in tracking of the OCT data acquisition. (See, for example, U.S. Pat. No. 5,537,162, US20070291277, & US20120249956; these are hereby incorporated by reference).

In one embodiment of this application, a scan of a large field-of-view of the fundus is obtained using the fundus imaging system (a first imaging modality). An example of such a fundus image is shown in FIG. 3. This image is then automatically processed using algorithms (see, e.g., Deckert et al. 2005) to find regions-of-interest (301). Manual selection of a region-of-interest (303) is likewise possible. These regions-of-interest could be normal structures such as the fovea or the optic disc. They could also be any pathological regions, e.g., drusen or geographic atrophy (GA) areas. Fast automated analysis of the fundus image enables the accurate localization of the regions-of-interest like the ones indicated by the region enclosed by the dashed line (302) in FIG. 2.

In this particular embodiment, information thus obtained can be used to control the scan of a second imaging modality (e.g., OCT) of these regions-of-interest. The scan parameters of the second imaging modality could be changed based on the information provided by first imaging modality such as extent of the pathology. The embodiments proposed herein are for the automatic determination via processing of the following scan parameters.

FIG. 4 presents another example of using one modality to supplement the information content derived from another. In FIG. 4a, a large area FA fundus image is shown of the fundus of an eye of a patient beset with diabetic retinopathy. FIG. 4b presents a small area image, taken with functional OCT, of the foveal avascular zone (FAZ). With this latter technique, the FAZ can be followed over time without contrast agents or injections (with known toxic fluorophores as discussed above).

Scan parameters may consist of any of the following: axial resolution, lateral resolution, strength of light signal, scan depth, over-sampling factor, locations, field-of-view, depth-of-focus, position of best axial focus, and focal ratios. The over-sampling factor is defined to be the ratio of the beam diameter to the lateral step size or increment. In the case of FA movie or OPMI-display, the scan parameter to be communicated also includes parameters to realize visual references in the live display such as superimposed segmented vessels or tumor volumes in 2D or 3D.

In an example, a region-of-interest is selected from within a fundus imaging by the rectangular box (303) in FIG. 3. Automated analysis of a first imaging modality (in this example, fundus imaging) for finding the region-of-interest or regions-of-interest might include feature extraction such as blood vessel segmentation, optic disc segmentation, and fovea segmentation. (Optical nerve head and optic disc are synonymous terms.) Regions-of-interest might be extracted based on intensity analysis and/or texture analysis as would be known to one skilled in the art (see, e.g., Iyer et al. 2006 and Iyer et al. 2007).

The expected locations of certain lesions might be initialized by the segmentation or quick location of the anatomical features such as the optic nerve head and fovea. For example, geographic atrophy usually occurs around the foveal region and peripapillary atrophy occurs around the optic disc/optic nerve head. The approaches described herein use an alternate imaging modality to locate the regions-of-interest which has the advantage that it can precisely define features of interest even in pathological cases that can be subsequently imaged again, but with an alternative modality.

For example, in cases where the fovea is severely disrupted due to edema, it might be difficult even to pinpoint the location of the fovea looking at the OCT data. However, using the information of the blood vessel arcades and the optic disc derived from a fundus imaging, it will be possible to locate the fovea accurately and then place the OCT scan over that region. The system could also detect multiple regions-of-interest for the same eye and guide the acquisition of multiple OCT datasets from these regions. It will also be possible to place the OCT scan based on different kinds of pathologies seen from different fundus imaging modalities. For example, a region of leakage could be visualized in an FA image and OCT imaging guided to the location of the leakage. Another example is visualization of GA using FA imaging and subsequent OCT imaging of the GA regions.

In another embodiment, it will be possible to change the field-of-view, sampling density and/or the lateral resolution (or other scan parameters) used in the second imaging modality based upon the extent of the region-of-interest that was detected using the first imaging modality.

For example, if a large geographic atrophy or area of pathological disturbance is detected from the fundus imaging, then it will be possible for the system to automatically change the field-of-view of the OCT image so that it captures the whole region of the pathological disturbance. The lateral or transverse (x,y) resolution of the OCT image could be adaptively changed based on a tradeoff between the field-of-view and the length of time desired for the scan. The axial resolution can also be so altered to optimize the information content of the derived image. For instance, standard OCT scans cover a region of 6 mm×6 mm around the fovea. It is, however, sometimes seen that the GA extends out of this central square region. Depending on the GA detected from the fundus imaging modality, the scan region of the OCT could be changed for example to be 9 mm×6 mm (assuming the GA extended horizontally): assuming a standard 6 mm×6 mm scan is composed of 200 B-Scans with 200 A-Scans/B-scan. In the scenario mentioned above, we could either scan the region with the same 200 B-Scans (same vertical area of the scan) and increase the number of A-Scans per B-Scan to 300. This will result in the final 9 mm×6 mm OCT scan having the same lateral resolution as the original 6 mm×6 mm cube. However in this case, the acquisition time would approximately increase 1.5 times. Another alternative is to keep the number of A-Scans per B-Scan constant but scan the larger 9 mm area. In this case the resolution of the OCT along the x-dimension would degrade.

Another embodiment is to change the OCT resolution adaptively around regions-of-interest. In the case of a foveal scan, the highest resolution is desired near the fovea while the scan may be more sparsely sampled progressing into the periphery, where the information content may be of lesser importance. Thus using the information content derived from the image of the first imaging modality, the OCT scan resolution or OCT control parameters can be changed adaptively or dynamically. This idea can be further expanded to obtaining multiple smaller FOV OCT scans with at least two different sampling densities and combing these individual OCT scans to create a larger FOV data set. The method to have densely sampled OCT data near the fovea and sparsely sampled OCT data in the periphery can be especially useful in functional OCT imaging techniques such as OCT angiography. For example, the choriocapillaris layer network is more dense near the fovea compared to the periphery and hence it would be beneficial to perform denser OCT acquisition at the fovea compared to the periphery. (See, e.g., Choi et al. 2013.)

In an extension of the above embodiment, multiple smaller field-of-view (FOV) OCT scans with variable scanning density can be combined to generate a larger FOV 3D OCT or functional OCT data set. Also there are some pathologies such as micro-aneurysms that can be visualized better with increased sampling density, whereas pathologies such as ischemia or vein occlusions may require larger FOV scans with perhaps sparser sampling.

In an embodiment of the present application, a method is given that uses fundus imaging information from an imaging method other than OCT (e.g., laser scanning ophthalmoscope) to detect the location of the optical nerve head center and use this information to direct acquisition of high-density circular scans around the optic nerve head. Alternatively, an accurate location for the center of the optic nerve head can be derived from a 3D OCT data acquisition assuming tracking mode has been enabled. Upon discernment of the location of the optical nerve head one or more high-density scans about that location can be acquired. The RNFL thickness measurements can be obtained by segmentation on the averaged circular scan with high data quality, as can the other retinal layers that exist between the ILM and Bruch's membrane.

In another embodiment, the region-of-interest could be selected based upon an alteration in the morphological or pathological composition of the fundus images. Change analysis derived from fundus images (taken at different times) enables detection of various vascular and non-vascular regions of change in the eye (see, e.g., Iyer et al., 2006, 2007). Such analysis would enable accurate identification of regions that are clinically interesting to merit OCT imagery. In current systems once an OCT scan is obtained, a “repeat scan” is usually placed at the exact same region as the old scan. However, in cases where interesting changes are occurring at other places, an aspect of the present application will direct the OCT to acquire data at the region-of-interest, as the OCT data from the previous visit might not have been acquired in that region. Once regions-of-interest have been located via automatic processing of one imaging modality, in this case fundus images, scan parameters can then be automatically determined. These can be stored and upon a repeat visit by a patient for subsequent examination, can then be recalled and used for re-imaging of the same regions-of-interest (or pathology) so as to be able to detect disease progression.

In another embodiment, a low resolution wide field OCT “spotter” scan is acquired and stored for each acquisition session of a patient. The spotter scans can be analyzed automatically to find features of interest—for example the retinal thickness at each point. The “spotter” scan from a subsequent session can be compared to the spotter scan from the previous session to quickly find regions of gross change. The OCT system can then be directed to acquire high resolution images over these regions-of-interest based on the registrations of the OCT images guaranteed by the tracking system.

In another embodiment, certain OCT instruments typically allow locating an OCT-scan (cubes or 3D volumes) such that it matches the location of a previously acquired OCT-scan to allow for precise comparisons and change analysis. For this approach a fast tracking system (fundus imaging-based) that matches the new scan location with that of the previously acquired OCT-scan would be appropriate. Small field-of-view OCT scans by themselves are less likely to provide sufficient landmarks for adequate registration and hence using information from a different modality such as fundus imaging with a wider field-of-view can provide geographic guidance. (A landmark within the eye is defined to be one of those structures that are always present in the retina of an eye, such as fovea, macula, optic nerve head, medium to large vessels, and vessel crossings.)

In clinical IT-systems such as EMR (electronic medical records), modality worklists (MWL) transmit information from patient management and review terminals to acquisition devices to transfer patient information and work-instructions to speed up the work-flow by minimizing the need for entering information at the acquisition devices. However, the operator still needs to choose and position scans to generate the required information. Various imaging modalities are often controlled by an imaging control station. Such a station could be remote from the instrument itself, controlled via a server system, or be located remotely or controlled by a remote client. A station is considered ‘remote’ if it is not physically connected to another component that is involved with image acquisition. This means that the remote imaging control station could be in the same room, same enclosure, or even in another part of the world.

The steps of one embodiment of the present invention that overcomes the aforementioned difficulty may be summarized as follows and reference is made to FIG. 5:
1. A fundus instrument (C1) is used to obtain a fundus imaging, which is then is analysed (manually or algorithmically) using a review terminal R1 in which one or more regions-of-interest (ROIs) are identified and marked. This could be done automatically as previously described or manually based on input therefrom.
2. An OCT scan type is chosen, or one is automatically recommended, that intersects the ROIs in an optimal way. An example would be high-definition B-scan through the “center of gravity” of a pigmentation change and through the optical fovea as a geometric reference. (See Sander et al. 2005; Szkulmowski et al. 2011, for an explanation of high-definition B-scan.)
3. The fundus imaging or a processed representation of same is transmitted to a capture terminal (C2), as well as the ROI and the chosen scan type. An example of a capture device (C2) is an OCT-instrument or a controlling processor. The pre-processing step could be an extraction of geometric features like vessels and/or the ONH either by segmentation or by some geometric centering algorithm. The reference image, ROI, and scan-type could be transmitted as a package with the modality work list (MWL) through an EMR-system to a clinic.
4. The capture terminal (C2) uses the received data to position and perform the required OCT-scan with minimal interaction of the operator. An example would be automatic patient alignment could allow that the operator only confirms patient identity. The capture station (C2) then uses a system providing real-time information (OCT real-time image, LSLO or real-time OCT low resolution image that substitutes for an LSO image) to match retina position to the reference image. The chosen OCT-scan is then taken of the ROI(s).
5. The system can automatically provide a quality check of the acquired data to exclude distortions (for example cataract, blinking, non-optimal delay and polarisation settings, pupil misalignment) and to ensure sufficient landmark quality (for example by degree of landmark correlation). Subsequent re-alignment/acquisition (automatic or by user interaction) to improve quality is an option.
6. The acquired OCT-data can transmitted from the capture terminal (C2) to the EMR-system to allow its review together with the associated fundus information at a review terminal (R2). (The capture and review terminals may be located within the same system.)

This procedure allows for high-definition line scans that are positioned at locations of abnormalities as found in fundus imaging. Moreover it provides precise position of OCT-scans at regions-of-interest that are associated with changes in fundus images using devices (such as capture terminals) that are not amenable to real-time fundus imaging acquisition and/or fundus imaging acquisition in the same spectral region that was used for the identification of the regions-of-interest.

This particular embodiment allows a clinician or an automatic algorithm to review and evaluate the results. Currently, commercially available OCT systems provide a variety of scan patterns for users to choose from. For example macular scans centered on the macula and optic disc scans centered on the optic disc can be selected depending on clinical information desired. Each type of scan pattern will only support a particular subset of analysis capabilities like retinal nerve fiber layer (RNFL) segmentation or inner limiting membrane-retinal pigment epithelium (ILM-RPE) segmentation. The user usually has to manually select each scan type and then place the scans at the location of interest. Because of the need for acquiring different scan types separately, there is considerable amount of time spent by the users in acquiring the OCT data of interest. The current invention aims to automate much of this and help to avoid the user having to manually select and acquire different scan types.

The speckle reduced tomograms or B-scans allow the doctor to see the layers, morphology, and disruptions in detail with reduced noise and enhanced contrast, while the cube scans allow algorithms to act in three dimensions. There is also the possibility of registering the 2D scans to the 3D scan, where the doctor can see the 2D picture in the context of where particular layers are, or the doctor can focus on areas of interest identified in algorithms acting on the 3D data. There is also the possibility of using the 2D scans with better signal and reduced noise to inform analysis on the cube.

An embodiment of the present introduces a new scan pattern for OCT devices with a wider field-of-view volume, extensive analysis capabilities, variable number of embedded high-definition (HD) scans and automatic high-definition (HD) line placement based on automatic analysis of multiple information sources. The main use of the new scan pattern will be with newer higher speed and/or tracking enabled OCT systems in which significant cubes of data can be acquired without the negative impacts of motion. The scan pattern could be the “one” and only scan pattern that is needed and will provide quantitative and qualitative information about the macula, optic disc and other pathologies of interest.

The main components of a preferred embodiment of the new Mega Scan pattern are the following:
1. A wide field OCT cube scan with a minimum field of view of 12 mm×12 mm that contains both the macular and optic disc regions
2. Automatically generated analysis including but not limited to:
   a. ILM-RPE segmentation
   b. RNFL Segmentation
   c. Ganglion cell complex (GCC) Segmentation
   d. Other retinal layer segmentation
   e. Optic disc detection
   f. Optic Nerve Head segmentation
   g. Fovea detection
   h. Automatic ETDRS grid placement and retinal thickness measurements
   i. Automatic extraction of RNFL thickness around the optic disc
3. High-Definition (HD) Line Scans with speckle averaging embedded in the cube. The number of high definition (HD) scans can be fixed or variable based on automatically identified parameters.
4. The location of the HD scan placement is automatically determined based on
   a. Segmentation of Regions-of-interest from scanning laser ophthalmoscopes (SLO)/line scanning ophthalmoscopes (LSO) images
   b. Segmentation of Regions of interest from OCT scout scans (very low resolution cube scan at the beginning)

The adjustment of the OCT scan parameters (enumerated above) can either be automatic (meaning algorithmic) or via a clinician or operator. There is the option to provide real-time retinal imagery for the ready identification of landmarks. Once this has been accomplished, then the reference and real-time data landmarks can be matched using any standard technique such as cross-correlations. Once the positioning or alignment has occurred imaging can take place. Alignment of reference and real-time images will also have to account for scale or magnification changes. Again, this can be determined by standard techniques used in image matching. (See, e.g., Biomedical Image Registration, 2006.) This is particularly important, as the reference landmarks might have originated from different systems such as a fundus imaging modality. As eye length and refractive error might change the relative scales on different systems, it is best to perform magnification matching and rotation/translation on the acquisition device after landmark identification. (See, e.g., Matsopoulos et al. 2004; Stewart et al. 2003.)

In order to transmit scan parameters from one station to another, information about the first and second stations (e.g., different fields-of-view) need to be evaluated so that a sufficient transformation or projection of the image, taken with one station, can be forwarded to the new station. This transformation or projection procedure can naturally be accomplished automatically by image matching methods such as 2D/3D cross-correlation techniques, well known in the art.

If the precision of the acquisition is sufficient, then no post-processing registration will be necessary. Nevertheless, post-processing registration would help to increase matching of fundus imagery with that of OCT. For this, storing the closest-in-time real-time OCT data with the desired high-definition B-scan would be beneficial. Furthermore, OCT scans could be positioned with respect to stereo fundus images. The positioning could then be in 3D instead of 2D. During the positioning activity, the review station can show the spatial representation of the OCT scan.

It is apparent that the reference image, from some fundus Imaging modality, needs to be of sufficient quality so that the correlation of its landmarks with those of the OCT image can yield a reliable correlation peak. A quality assessment, e.g., with respect to the quality of focus and/or contrast, prior to the start of automatic or human evaluation could aid in avoiding failure of landmark correlation during later acquisition.

Should the evaluated fundus imaging and OCT image lack a common landmark or it is not sufficiently visible in one or both of the images, then correlating with data of another modality can be performed, if these data possess landmarks present in both the fundus imaging and in the OCT image. For example, a suspicious feature is noticed in a blue fundus image. This blue fundus image is correlated with a green or RGB-fundus image. The green image is then correlated with a red fundus image or the red part of the RGB fundus image. This latter image can be used to identify landmarks that can be correlated sufficiently with the 850 nm image from the OCT system.

To accelerate OCT real-time image acquisition, the scan location can be chosen such that the more identifiable landmarks are imaged with the highest probably and at locations that minimize errors in correlating landmarks (e.g., circle around the ONH with sufficient distance to keep rotational error small). Instead of obtaining OCT imagery, any of the fundus imaging modalities can also be used.

The gold standard for diagnosing defects of the optic nerve head and retinal nerve fiber layer typical of glaucomatous optic neuropathy is stereo fundus imaging. Recent advances in three dimensional analysis of OCT data have proven to be similar to the standard evaluation in terms of identifying the borders of the optic disc and the neuroretinal rim tissue, and provide the additional benefit of providing quantitative and reproducible information about the peripapillary retinal nerve fiber layer (RNFL). However, some characteristics of glaucomatous damage, including pallor of the disc and disc hemorrhages, cannot be appreciated in OCT images.

Prata et al. (2009) have shown that glaucomatous progression occurs preferentially near hemorrhages (vascular leakage), so it is reasonable to imagine that an analysis that examines OCT data for damage to the nerve fibers near a hemorrhage would allow earlier detection of progressive damage or that an analysis that combines information from several imaging modalities would allow improved evaluation of the risk of progression or staging of disease. Note that in this case the location of the pathology detected in one modality does not have to be identical as the location imaged by the other modality. For example, disc hemorrhage is located specifically near the optic disc, but may be associated with a wedge defect in the retinal nerve fiber layer that follows the arcuate path of the damaged axons. The progressive damage could be detected or monitored on OCT in an area that is angularly related to the hemorrhage but not in the same exact location. A Bayesian approach could be used (see, e.g., Sample et al. 2004), with the disc hemorrhage giving an increased prior estimation of the likelihood of progression in an angular region of the OCT circle scan that is related to the location of the hemorrhage relative to the disc, thereby increasing the post-test likelihood of progression even in a case where a small amount of change occurs.

In another embodiment, the fundus imaging can be used to identify pigment abnormalities which may or may not correspond to retinal pigment epithelium elevations detected by OCT analysis. Furthermore, OCT can show elevations of retinal pigment epithelium that are difficult to appreciate in fundus imaging. The reliability of automatic algorithms (Lee et al. 2012) to segment and to quantify elevations in the retinal pigment epithelium has recently been demonstrated in patients with age-related macular degeneration and other diseases as well (Smretschnig et al. 2010; Ahlers et al. 2008; Penha et al. 2012).

Label-free fundus imaging techniques (that do not require injection of a dye into patients) have been developed to do functional imaging such as blood flow (see, e.g., Tam et al. 2010). They obtained a series of adaptive-optics-based SLO images of the retina and applied motion contrast techniques to enhance the blood flow in parafoveal capillaries. Hiroshi Imamura (U.S. Pat. No. 8,602,556) proposed using a SLO/OCT multimodal system, where SLO imaging is used to identify retinal vasculature information and OCT is used to obtain depth information of the corresponding vasculature identified by SLO images. Imamura talks about use of structural OCT information alone to identify the depth of vessel. Ferguson et al. (2004) used scanning line ophthalmoscope and did temporal signal change analysis to obtain retinal perfusion and vascular flow images. Functional OCT based motion-contrast techniques offer depth-resolving capability advantages over 2D fundus imaging based motion-contrast techniques. However, one of the limitations of OCT angiography techniques is the longer acquisition times due to dense data sampling. But if the region of interest can be identified or narrowed down, then this information can be used to perform OCT angiography scans in the region of interest. In this embodiment we propose use of obtaining functional information using a fundus imaging modality by doing a motion contrast or change analysis (see, e.g., Ferguson et al. 2004; Tam et al. 2010; U.S. Pat. No. 8,602,556; Fischer et al. 2012), use the results of the change analysis to identify regions of interest and using this information to aid in acquisition of functional OCT acquisition. In one of the practical applications of such a method, fundus imaging can provide larger FOV functional or blood flow images of the eye (at least 25% greater coverage than the subsequent imaging modality), and then OCT could be used to obtain higher resolution image based on the ROI selected based on the larger FOV image from the first imaging modality. In addition, there could be a combined analysis wherein the correspondences are derived between functional information from fundus imaging modality and functional information from OCT.

The approaches described herein either involve the prior analysis of one modality to guide obtaining a second image from a distinct imaging modality or a simultaneous analysis of the information content derived from both modalities. Regardless of the particular approach that is taken, classifications would be based on features extracted from the images, such as image intensity relative to a reference/geographic point or perhaps by local variability in image intensity.

### Metrics and Characteristic Information

A combination of information (characteristic information) derived from OCT images and fundus imaging (including angiography), with one or more images are analyzed from each of the sources in which ROIs have been identified, can lead to metrics (characteristic metrics) for each pathology in itself. Also these individual characteristic metrics can be combined to derive a metric or estimation for the risk or likelihood of disease progression or severity of disease, or an estimation of the likelihood of the presence of disease. These ROIs may be classified, e.g., according to the lesion or pathological type, risk of pathology, risk of progression of pathology, etc.

Alternatively, instead of delivering classifications (or metrics) for each region or each imaging modality, an overall classification may be composed for the eye/subject that is derived either based upon a combination of the metrics obtained for each individual morphological/pathological condition or by deriving a single metric based upon analysis of the ensemble of clinical imagery.

Such metrics characteristic of the information derived from a specific imaging modality may include: RNFL thickness or progressive thinning of the RNFL (i.e., rate-of-change) or other observables of other retinal layers, cup-to-disc ratio, total area or volume of intra-retinal or sub-retinal fluid, drusen characteristics such as reflectivity, area, volume, pigmentation variations or some characterization of content such as primarily fibrovascular or primarily serous, extent of geographic area, characteristics of the border around GA, including disturbance of the IS/OS, neuroretinal rim thickness, metrics of vascularization including vessel density or tortuosity, numbers of micro-aneurysms, area of photoreceptor disruption, as well as pallor, and abnormalities in coloration. (Pallor in this application refers to the nature of vascular perfusion in an area of the eye.) Weighting the intensity with radial moments from a midpoint location, and deriving a characteristic radius can then be used to monitor chronological progression.

In any of the fundus or OCT imaging modalities, pattern recognition (see, e.g., Fukunaga 1990; Bishop 2006) and classification is used to locate and to characterize the extent of the abnormalities. Extent in the context of the present application refers to either areal (2D) or volumetric (3D) measures and the context will be obvious to the ordinary skilled person in the art. An area can be derived from any fundus imaging modality and or any enface projection of a volumetric data set from 3D to 2D. A volumetric extent is derivable by combining an areal extent with knowledge of the depth under than area, which is derivable only from OCT measurements. Moreover, with repeated measurements concomitant with appropriate drug therapies, OCT can provide a guide for the adjustment of the therapeutic dosages.

A metric can be derived from the aforementioned components of the OCT characteristic information by at least a weighted combination. Naturally, many would have to be placed in context defined by a normative database. Appropriate processing of the images can yield information about pathological features such as location, thickness, extent, and frequency. Moreover, by processing the data from one modality, guidance information can be derived to permit efficient imaging and information derived therefrom by another modality. An example would be using the information derived from a fundus imaging to determine a region-of-interest to image using an optical coherence tomographic system. For instance, suspected vasculature related pathologies could be identified using fundus Imaging, and suspected regions could later be scanned by OCT to generate functional information such as blood flow. Thus this could be accomplished either in a single multimodality imaging system or via a plurality of imaging systems connected via network.

Characteristic information derivable from fundus imaging modalities include, but are not limited to: extent of drusen, geographic atrophy, hard and soft exudates, cotton-wool spots, blood flow, ischemia, vascular leakage, reflectivities as a function of depth and wavelength; hyper- or hypo-pigmentation abnormalities (often due to the absence of melanin or the presence of lipofuscin); colors based on relative intensities at different wavelengths; and chronological changes in any of these. The extent of many of these observables is directly correlated with the likelihood of the presence of disease, as is well known in the art. A metric of the likelihood of the presence of disease can then be determined, even in an automatic approach, by a weighted combination of the individual characteristics. Naturally, each component of the characteristic information would be relative to that of a normative database.

Classification of stage of disease, probability of disease, risk of progression of disease, an estimation of the likelihood of disease presence, or an estimation of the likelihood of progression could also be made based on a combination of image features from distinct imaging modalities. Such features would be derived at each lateral position and decisions about each point would be based upon a comparison of these features to limits empirically determined by comparison to normal or diseased eyes. The mechanism for the decisions may be simple Boolean logic, linear or nonlinear discriminant analysis, or more complex neural or fuzzy system classifiers. (See, e.g., US20120274898; Fukunaga 1990; Bishop 2006.)

The structure of retinal vasculature can provide valuable information regarding the state of disease within the eye. It has been noted that retinal vessel caliber is an early indicator of cardiovascular disease (Wong et al. 2002). The caliber is indicative of hypertension, proliferative diabetes, arteriosclerosis, and other cardiovascular-related diseases (Xiaofang et al. 2010). Studies have demonstrated that subtle changes occur in the retinal vasculature such as arteriolar to venular ratio, focal abnormalities of arterioles, and arteriolar/venular crossing abnormalities, diminished branching angle at bifurcations (indicative of endothelial function), increase arteriolar length-to-diameter ratio, and a reduced microvascular density. An overall descriptor of the results of these changes is that of tortuosity. Angiogenesis, excess of VEGF, microaneurysms, are all aspects of the result of retinal diseases (including diabetic retinopathy) and are correlated with tortuosity (Witt et al. 2006). Endothelial cells play an important function in the creation of angiogenesis and in the maintenance of microvascular blood flow. Increased flow results in increased tortuosity (Yamakawa et al. 2001). Thus metrics are derivable from physical phenomena such as vessel tortuosity, vessel width, vessel branching patterns and angles, venous beading, focal arterial narrowing neovascularization, fractal dimension of vasculature, and extent of micro-aneurysms.

Several metrics have been developed to measure the nature of the geometric configuration of the retinal vasculature. Some are defined by geometric properties (see, e.g., Hart et al. 1999; Hughes et al. 2006; Hao et al. 2013; Aliahmad et al. 2011), or from metrics derived via fractal analysis (see, e.g., Azemin et al. 2011; Thompson et al. 2008; McLean et al. 2002; Masters 2004). These metrics as well as others (see, e.g., Witt et al. 2006) can be used as indicators of the presence of disease, and when multiple temporally-disparate datasets become available, then a risk or likelihood of the progression of disease can be determined and reported. In addition, the efficacy of treatment options can be monitored with these chronological distinct datasets.

Philip et al. (2007) have derived a binary classification system based upon an algorithmic approach. While not a graded metric, at least this can separate diseased eyes from health ones. Candidate bright and dark lesions were identified by image analysis and features classified by a neural network.

### REFERENCES

### Non-Patent Literature

Iyer et al. 2006, IEEE Transactions on Bio Eng 53.6, 1084-1098.
Iyer et al. 2007, IEEE Transactions on Bio Eng 54.8, 1436-1445
Lee 2012, Inves Ophth Vis Sci 53, 164-70.
Stetson et al. 2013, Inves Oph Vis Sci 54, ARVO E-Abstract 6296.
Brar et al. 2009, Am J Ophthal 148, 439-444
Huang et al. 1991, Sci 254, 1178
Nassif et al. 2004, Opt Lett 29, 480.
Choma et al. 2003, Opt Exp 11, 2183
Wojtkowski et al. 2005, Ophthal 112, 1734.
Lee et al. 2006, Opt Exp 14, 4403
Leitgeb et al. 2004, Opt Exp 12, 2156
Atchison et al. 2004, IOVS 45, 3380-3386
Smretschnig et al. 2010, Graefes Arch Clin Exp Ophthal 238, 1693-1698
Ahlers et al. 2008, Br J Ophthal 92, 197-203
Penha et al. 2012, Am J Ophthal 153, 515-523.
Lee et al. 2012, Invest Ophthal Vis Sci 53, 164-170.
Prata et al. 2009, Ophthalmology 117, 24-29.
Pemp et al. 2013, Graefe's Arch Clin Exp Ophth 251, 1841-1848.
Hee et al. 1995, IEEE Engineering in Medicine and Biology, January/February
Wang et al. 2007, Opt Exp 15, 4083-4097.
An & Wang 2008, Opt Exp 16, 11438-11452.
Fingler et al. 2007, Opt Exp 15, 12636-12653.
Fingler et al. 2009, Opt Exp 17, 22190-22200.
Mariampillai et al. 2010, Opt Lett 35, 1257-1259.
Abramoff et al. 2008, Diabetes Care 33, e64.
Abramoff et al. 2010, IEEE Trans Biomed Eng. 3, 169-208.
Sayegh et al. 2011, Ophthal 118, 1844-1851.
Antal & Hajdu 2013, Comp Med Imag Graph 37, 403-408.
Sopharak et al. 2013, Comp Med Imag Graph 37, 394-402.
Iyer et al. 2013, U.S. provisional app. 61/785,420.
Deckert et al. 2005, BMC Ophthal 5:8, 1-8.
Salam et al. 2012, in Optical Coherence Tomography, Eds: Bernardes & Cunha-Vaz, Springer: Heidelberg.
Sander et al. 2005, Br J Opthal 89, 207-212.
Szkulmowski et al. 2011, Opt Exp 20, 1337-1359.
Tam et al. 2010, Inves Ophthal Vis Sci 51, 1691-1698
Azemin et al. 2011, IEEE Trans Med Imag 30, 243-250.
Hart et al. 1999, Int J Med Info 53, 239-252.
Thompson et al. 2008, J Neurol Neurosurg Psych 79, 448-250.
McLean et al. 2002, J Neurol Neurosurg Psych 72, 396-399.
Hughes et al. 2006, J. Hypertens 24, 889-894.
Aliahmad et al. 2011, IEEE EMBS, 33rd Ann Int Conf, 2606-2609.
Masters 2004, Ann Rev Biomed Eng 6, 427-452.
Hao et al. 2013, IEEE Conf Biosig Biorob, 1-4.
Yamakawa et al. 2001, Curr Eye Res 22, 258-265.
Wong et al. 2002, JAMA 287, 1153.
Xiaofang et al. 2010, ICCAE 2^{nd}, 443-446.
Witt et al. 2006, Hypertension 47, 975-981.
Gregori et al. 2011, Ophthalmology 118, 1373-9.
Yehoshua et al. 2013, Ophthalmic Surg Lasers Imaging Retina. 44, 127-32.
Matsopoulos et al. 2004, IEEE Trans. Med. Imag. 23, 1557-1563.
Stewart et al. 2003, IEEE Trans. Med. Imag. 22, 1379-1394.
Fukunaga, K. 1990, Introduction to Statistical Pattern Recognition, Academic Press.
Bishop C. M. 2006, Pattern Recognition and Machine Learning, Springer.
Sample et al. 2004, Invest Ophthal Vis Sci 45, 2596-2605.
Choi et al. 2013, PLoS One 8(12), e81499.
Fischer et al. 2012, PLoS One 7(4), e36155.
Biomedical Image Registration, 2006, 3rd International Workshop, Eds. Pluim, Likar, and Gerritsen.
Ferguson et al. 2004, Opt. Exp. 12, 5198-5208.

### REFERENCES

### Patent Literature

U.S. Pat. No. 8,602,556
US20120274898
US20110275931
US20120150029
U.S. Pat. No. 8,433,393
U.S. Pat. No. 5,321,501
U.S. Pat. No. 5,537,162
U.S. Pat. No. 7,301,644
U.S. Pat. No. 7,805,009
U.S. Pat. No. 7,884,945
US20070291277
US20120249956
U.S. application Ser. No. 13/354,066
U.S. Pat. No. 7,830,525
U.S. Pat. No. 6,095,648
US20110102802.
U.S. application Ser. No. 13/803,522
US 20080025570
US20070216909
US20070103693
US 20080025570 (Fingler et al. 2008)
US 20120274898 (Sadda and Stetson)
US20110190657 (Zhou et al. 2011)

1. A system to image an eye of a patient, comprising:
a first imaging modality for imaging the eye;
a second imaging modality for imaging the eye, said second imaging modality distinct from said first imaging modality;
a processor for analyzing one or more images from said first imaging modality to derive a region-of-interest and/or a set of scan parameters for the second imaging modality; and,
a controller for using said set of scan parameters or said region-of-interest to acquire one or more images using said second imaging modality;
wherein one of the imaging modalities is a functional imaging modality.

2. A system as recited in claim 1, in which the first imaging modality is fluorescein angiography and the second imaging modality is a functional optical coherence tomography (OCT) imaging modality.

3. A system as recited in claim 1, in which the first imaging modality is fundus autofluorescence and the second imaging modality is OCT.

4. A system to image an eye of a patient, comprising:
a first station for collecting images from a first imaging modality;
a second station for collecting images from a second imaging modality, distinct from said first imaging modality;
a processor for analyzing one or more images from said first imaging modality to derive a set of scan parameters for the second imaging modality; and
a controller for communicating said set of scan parameters to the second station and for using said set of scan parameters to control the acquisition of an image using said second imaging modality.

5. A system as recited in claim 4, in which said first imaging modality is a fundus imaging modality.

6. A system as recited in claim 4, in which said second imaging modality is selected from the group consisting of fundus imaging modalities, functional fundus imaging modalities, optical coherence tomographic systems, and functional optical coherence tomographic systems.

7-16. (canceled)

17. A method to image an eye of a patient, comprising:
collecting a first image of the eye with a first imaging modality;
collecting a second image of the eye with the first imaging modality at a subsequent patient visit;
identifying changes between the first and second images to determine a region-of-interest;
obtaining a third image of the eye containing said region-of-interest using a second imaging modality distinct from the first imaging modality; and,
displaying, storing, or further processing said third image.

18. A method as recited in claim 17, in which the first imaging modality is a fundus imaging modality and the second imaging modality is an optical coherence tomographic imaging modality.

19. A method as recited in claim 17, in which the identifying of changes is performed automatically.

20. A method according to claim 17, in which one of the imaging modalities is a functional imaging modality.

21. A method for imaging an eye of a patient, said method comprising:
collecting a first set of one or more images of the eye from an imaging modality;
processing automatically said first set of images to derive a set of scan parameters;
communicating said set of scan parameters to an imaging control station;
obtaining a second set of one or more images of the eye, in which the imaging control station controls the image acquisition using the scan parameters derived from the first set of images; and,
displaying, storing, or further processing said second set of images.

22. A method as recited in claim 21, in which the set of scan parameters are selected from the group consisting of axial resolution, scan depth, lateral resolution, strength of light signal, over-sampling factor, locations, fields-of-view, depths-of-focus, position of best axial focus, and focal ratios.

23. A method as recited in claim 21, in which the images of the first set and those of the second set have been obtained with the same imaging modality.

24. A method as recited in claim 21, in which the images of the first set and images of the second set have been obtained with distinct imaging modalities.

25. A method as recited in claim 21, in which the imaging control station is remote from said processor.

26. A method as recited in claim 21, further comprising storing the scan parameters and recalling them for repeat patient examinations so as to be able to detect progression of disease.

27. A method as recited in claim 23, in which the imaging modality is an optical coherence tomographic system, the first set of images is a 3D volume of OCT data, and the second set of images include one or more high-definition B-scans.

28. A method as recited in claim 27, further comprising:
processing said 3D volume or high-definition B-scans with one or more processing steps, in which the processing steps are selected from the list consisting of ILM-RPE segmentation, RNFL segmentation, ganglion cell complex (GCC) segmentation, retinal layer segmentations, optic disc detection, optic nerve head segmentation, fovea detection, automatic ETDRS grid placement, retinal thickness measurements, and automatic extraction of RNFL thickness around the optic disc;
reporting results from said processing steps; and,
storing, displaying, or further processing said volume and/or said high-definition B-scans and/or said results.

29. A method as recited in claim 27, in which the high-definition B-scan or scans are obtained by scanning laterally across the eye.

30. A method as recited in claim 27, in which the high-definition B-scan or scans are obtained by scanning the eye in a circular pattern.

31. An optical coherence tomographic (OCT) imaging system for collecting data from an eye of a patient, the system comprising:
a light source for generating a light beam propagating along an axis;
a beam divider for directing a first portion of the light beam into a reference arm and a second portion of the light beam into a sample arm;
optics for scanning the light beam in the sample arm over the eye to a plurality of positions in a plane perpendicular to the propagation axis of the beam;
a detector for measuring light radiation returning from the sample and reference arms, and generating output signals in response thereto;
a processor for analyzing a retinal image to determine a set of parameters for use in scanning the light beam in the sample arm over the eye; and,
a controller for scanning the light beam using the set of parameters.

32. A system as recited in claim 31, in which the set of parameters are selected from the group consisting of axial resolution, scan depth, lateral resolution, strength of light signal, over-sampling factor, locations, fields-of-view, depths-of-focus, position of best axial focus, and focal ratio.

33. A system as recited in claim 31, further comprising a secondary imaging modality for collecting retinal images, and wherein the processor analyzes the retinal images from the secondary imaging modality to determine a set of scan parameters.
